(19) United States
(12) Patent Application Publication
(10) Pub. No.: US 2010/0105943 A1
(43) Pub. Date: Apr. 29, 2010

(54) HYPERBRANCHED POLYESTERS

(75) Inventors: Daniel Schoenfelder, Mannheim (DE); Bernd Bruchmann, Freinsheim (DE); Robert Feuerhake, Mannheim (DE); Hans-Joachim Haehnle, Neustadt (DE); Joachim Pakusch, Speyer (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/532,493

(22) PCT Filed: Mar. 14, 2008
(86) PCT No.: PCT/EP2008/053086

§ 371 Date: Sep. 22, 2009

(30) Foreign Application Priority Data
Mar. 22, 2007 (EP) 07104693.2

(51) Int. Cl. C07C 69/66; C07C 69/76; C07C 69/73; C07C 229/00

(52) U.S. Cl. 560/76; 560/180; 560/181; 560/171

(57) ABSTRACT

The present invention relates to new hyperbranched polyesters based on oligohydroxycarboxylic acids with defined functionality.

The present invention relates to new hyperbranched polyesters based on oligohydroxycarboxylic acids with defined functionality.

Hyperbranched polyesters based on dimethylolpropionic acid are known, for example, from EP 630389 B1, WO 00/222700 or EP 1582548A.

A disadvantage of these hyperbranched polyesters, however, is that they are prepared under relatively indefinite reaction conditions, and so have nonuniform functionalities.

Perfectly dendrimeric polyesters comprising dimethylolpropionic acid units are likewise known, for example, from E. Malmström et al., Macromolecules 2004, 37(2), 322-329.

A disadvantage of these compounds is that in the synthesis it is not possible to use dimethylolpropionic acid but instead, in order to achieve the high selectivities, dimethylolpropionic acid bearing an acetonide as protective group is used.

Consequently a synthesis of this kind necessitates on the one hand the not inexpensive acetonide-protected dimethylolpropionic acid as a starting component and, on the other hand, reaction steps for removing these protective groups.

The aforementioned publication also describes the need for hyperbranched polyesters of this type comprising copolymerizable groups, such as acrylate groups or methacrylate groups, for example, for use in the preparation of polymers, for example. In this case, it is important for such compounds to have precisely one free-radically polymerizable group, since compounds having two or more free-radically polymerizable groups would lead to crosslinking of the polymers.

There is therefore a need for hyperbranched polyesters which are easy to synthesize and which are substantially monofunctional in terms of free-radically polymerizable groups.

It was an object of the present invention to provide hyperbranched polyesters having a regular structure which can be prepared starting from simple synthesis units without protective-group operations, which are substantially monofunctional in respect of carboxyl groups, and which can easily be converted into compounds which are likewise substantially monofunctional and are, for example, free-radically polymerizable.

This object has been achieved by means of hyperbranched polyesters having a weight-average molecular weight M_{W} between 500 and 300 000 g/mol, obtainable by reacting at least one oligohydroxycarboxylic acid in the presence of at least one catalyst and in the presence of at least one solvent inert under the reaction conditions, wherein the temperature during the reaction is set at not more than 120° C., the average carboxyl functionality per molecule is more than 0.8 and less than 2, and the average ether group functionality —CH₂—O—CH₂— is less than 0.20.

Hyperbranched polyesters for the purposes of this invention are noncrosslinked macromolecules containing hydroxyl and carboxyl groups that possess both structural and molecular nonuniformity. On the one hand they can be constructed starting from a central molecule, in the same way as dendrimers, but with a nonuniform branch chain length. On the other hand, they can also have a linear construction with functional, branched side groups, or else may have both linear and branched moieties as a combination of the two extremes. On the definition of dendrimeric and hyperbranched polymers see also P. J. Flory, J. Am. Chem. Soc., 1952, 74, 2718 and H. Frey et al., Chem. Eur. J., 2000, 6 (14), 2499.

By “highly branched” and “hyperbranched”, in connection with the present invention, is meant that the degree of branching (DB), in other words the average number of dendritic linkages plus the average number of end groups per molecule, divided by the sum of the average number of dendritic linkages, the average number of linear linkages and the average number of end groups, multiplied by 100, is 10% to 99.9%, preferably 20% to 99%, more preferably 20% to 95%.

By “dendrimeric” in the context of the present invention is meant that the degree of branching is 99.9%-100%. On the definition of the “degree of branching” see H. Frey et al., Acta Polym. 1997, 48, 30.

An important feature of the polyesters is that they are noncrosslinked. “Noncrosslinked”, for the purposes of this specification, means that there is a degree of crosslinking of less than 15% by weight, preferably of less than 10% by weight, determined via the insoluble fraction of the polymer.

The insoluble fraction of the polymer was determined by extraction for four hours with the same solvent as used for the gel permeation chromatography, in other words a solvent selected from the group consisting of tetrahydrofuran, dimethylacetamide and hexafluoroisopropanol, depending on the solvent in which the polymer has the better solubility, in a Soxhlet apparatus and, after drying of the residue to a constant weight, the weighing of the residue that remains.

The synthesis reaction of this kind of polyester is accomplished by reaction of the carboxyl group of the oligohydroxycarboxylic acid with one of the hydroxyl groups. In an ideal synthesis reaction, by esterification of n molecules of an oligohydroxycarboxylic acid bifunctional in respect of hydroxyl groups, this would lead to a hyperbranched polyester having (n+1) hydroxyl groups and precisely one carboxyl group. In reality this ideal construction, however, is disrupted by etherification reactions of the hydroxyl groups and by intramolecular esterification (lactone formation).

The etherification reaction results in fewer than the ideal number of hydroxyl groups being present in the product. Ether groups formed intramolecularly have no consequences for the number of carboxyl groups per molecule. For each intramolecularly formed ether group, in contrast, a further free carboxyl function is formed in the hyperbranched polyester. This, however, is in contradiction with the stated requirement for monofunctional products of uniform construction. The etherification reaction must therefore by prevented or at least suppressed.

Lactone formation takes place through intramolecular reaction of a carboxyl group with a hydroxyl group, and as well as an irregular construction results in a reduction in the number of free carboxyl groups in the product. This reaction lowers the average carboxyl functionality of the product. This, however, is in contradiction with the stated requirement for monofunctional products of uniform construction. It is therefore necessary to prevent or at least suppress lactone formation. Since lactone formation lowers the carboxyl functionality of the molecule, whereas the intermolecular etherification increases it, lactone formation is to be tolerated more than a reaction which leads to an increase in the carboxyl functionality. Preferably, however, lactone formation as well should be as low as possible.

The weight-average molar weight M_{w} (determined by gel permeation chromatography using polymethyl methacrylate standards) of the hyperbranched polyesters of the invention is generally between 500 and 300 000, preferably from 600 to 200 000 g/mol; the number-average molar weight Mₙ is between 400 and 50 000, preferably between 500 and 30 000 g/mol.

The polydispersity is generally from 1.1 to 30, preferably from 1.2 to 20.

The high-functionality, highly branched or hyperbranched polyesters of the invention are liquid or solid at room temperature (23° C.) and generally have a glass transition temperature of −50 to 120° C., preferably of −40 to 100° C. and more preferably of −30 to 80° C. High-functionality, highly branched or hyperbranched polyesters of this kind, constructed from aromatic oligohydroxycarboxylic acids, can have a glass transition temperature of up to 180° C.

The glass transition temperature T_{g} is determined by the DSC (Differential Scanning Calorimetry) method in accordance with ASTM 3418/82, with a heating rate of preferably 10° C./min.

The OH number, according to DIN 53240, Part 2 of the hyperbranched polyesters is generally from 1 to 800 mg KOH/g, preferably from 50 to 800 mg KOH/g, more preferably from 80 to 700 mg KOH/g.

Essential to the invention is that the hyperbranched polyesters of the invention have an average carboxyl (COOH) functionality of more than 0.7, preferably more than 0.8, more preferably more than 0.9 and very preferably more than 0.95, and less than 2, preferably less than 1.8, more preferably less than 1.5, very preferably less than 1.3, more particularly less than 1.1 and especially less than 1.05.

The average functionality is the average number of the functional groups in question per molecule.

Unwanted macromolecules with different carboxyl contents can be detected particularly effectively by mass-spectrometric methods, such as by MALDI-TOF analysis. Thus, by adding different salts (e.g. with a cation of molar mass “cat” from the series of the Li⁺, Na⁺ or K⁺ salts) on sample preparation prior to the MALDI-TOF measurement, the signals of the carboxyl-containing macromolecules can be separated from the signal series of the other components without carboxyl groups. Macromolecules with carboxyl groups may, in addition to the customary signals of mass (M+cat)⁺, as a result of proton-cation exchange for every carboxyl group present, display additional signals, with a mass value increased by the mass of the cation reduced by one mass unit (cat−1). Hence for every macromolecule depending on its carboxyl functionality, a typical mass-spectrometric signal pattern is produced at a mass spacing of (cat−1).

The three examples below are intended to illustrate this situation:

in the case of macromolecules monofunctional in respect of the carboxyl groups, the only signals which appear in the MALDI-TOF are the cationized molecule ion (M+cat)⁺ and also the increased signal originating from proton-cation exchange at (M+(cat−1)+cat)⁺. Macromolecules having two carboxyl groups per molecule exhibit these two signal series plus a third series, which originates from dual proton-cation exchange and has a mass increased by 2*(cat−1):(M+2*(cat−1)+cat)⁺. Macromolecules without carboxyl groups, in contrast, would display only the main signal at (M+cat)⁺, since without a free carboxyl group it is not possible for proton-cation exchange to take place.

A further feature of the hyperbranched polyesters of the invention is that they have a low average ether group —CH₂—O—CH₂— functionality. This functionality is frequently less than 0.2, preferably less than 0.15, more preferably less than 0.10, very preferably less than 0.06 and more particularly less than 0.04. Ether groups of this kind are produced through inter- or intramolecular reaction of hydroxymethyl groups of units of the oligohydroxycarboxylic acid with one another. This results, in particular, in an irregular carboxyl group being formed for each intermolecular ether group.

The ether group —CH₂—O—CH₂— content of the product can be determined by means of ¹³C-NMR spectroscopy at 100 MHz in d₆-DMSO. Thus the CH₂ groups of the ether groups —CH₂—O—CH₂— are shifted downfield in ¹³C-NMR in comparison to the CH₂ groups of the ester groups —CH₂—O—OC—. For example, the CH₂ groups of the ether groups in the hyperbranched polyester of dimethylolbutyric acid appear approximately 2-10 ppm further downfield than the CH₂ groups of the ester groups, which appear at about 58-63 ppm.

A further feature of the hyperbranched polyesters of the invention is that they have a low lactone functionality. This functionality is generally less than 0.10, preferably less than 0.08, more preferably less than 0.06, very preferably less than 0.04 and more particularly less than 0.02. Lactones come about through intramolecular reaction of hydroxymethyl groups with carboxyl groups that have already undergone esterification or with carboxyl groups that are free. A particular consequence of the reaction with free carboxyl groups is that for each lactone one regular carboxyl group is broken down. Where, for example, two oligohydroxycarboxylic acid units react with one another, the product is a carboxyl-free, dimeric ring system. The reaction of correspondingly larger polyesters produces carboxyl-free macrocyclic lactones.

The lactone content of the product can be determined, for example, by means of two-dimensional NMR spectroscopy, as a result of transannular interactions of the hydrogen atoms.

Cyclic components of the hyperbranched polymer can likewise be detected in a particularly effective way by means of mass spectrometric methods, such as MALDI-TOF analysis, for example. For instance, hyperbranched polymers which comprise one ring per molecule, i.e. an intramolecular ether bond or an intramolecular ester bond (lactone), have a signal which is shifted by 18 mass units toward lower mass values in comparison to the main signal, since in comparison to the ring-free main signal they are produced as a result of the elimination of an additional molecule of water per macromolecule. Macromolecules with n rings per molecule, consequently, give a signal which is shifted by n * 18 mass units toward lower mass values as compared with the main signal.

The oligohydroxycarboxylic acids have not only precisely one carboxyl function but also at least two hydroxyl functions: for example two to five, preferably two to four, more preferably two to three, and very preferably precisely two.

The oligohydroxycarboxylic acids may comprise aromatic groups or may be aliphatic and preferably are aliphatic.

The hydroxyl groups may be present preferably in the form of hydroxymethyl groups (—CH₂—OH).

One example of compounds having precisely one carboxyl group which contain more than two hydroxymethyl groups in position 2 relative to the carboxyl group is 2,2,2-tris(hydroxymethyl)acetic acid. An oligohydroxycarboxylic acid of this kind which comprises hydroxyl groups in a form other than that of hydroxymethyl groups is 2,3-dihydroxypropionic acid, for example. Examples of compounds having precisely one carboxyl group and containing more than two hydroxyl groups are sugar acids such as gluconic acid, glucaric acid, glucuronic acid, galacturonic acid or mucic acid (galactaric acid).

Conceivable and preferred is the use of aromatic dihydroxycarboxylic acids such as 2,4-, 2,6- and preferably 3,5-dihydroxybenzoic acid or 4,4-bis(4-hydroxyphenyl)valeric acid.

Particularly preferred oligohydroxycarboxylic acids are 2,2-bis(hydroxymethyl)alkanecarboxylic acids.

Examples of 2,2-bis(hydroxymethyl)alkanecarboxylic acids that can be employed in accordance with the invention are those having five to twelve carbon atoms, preferably five to seven, more preferably six carbon atoms, such as, for example, 2,2-bis(hydroxymethyl)propionic acid (dimethylolpropionic acid), 2,2-bis(hydroxy-methyl)butyric acid (dimethylolbutyric acid) and 2,2-bis(hydroxymethyl)valeric acid, preferably 2,2-bis(hydroxymethyl)propionic acid (dimethylolpropionic acid) or 2,2-bis(hydroxymethyl)butyric acid (dimethylolbutyric acid) and with particular preference 2,2-bis(hydroxymethyl)butyric acid (dimethylolbutyric acid).

The oligohydroxycarboxylic acids can be used in the form of salts, as ammonium or alkali metal salts, for example, or can be used preferably as free acids.

In one preferred embodiment the reaction according to the invention is carried out in the presence of not more than 25 mol % (based on the amount of oligohydroxycarboxylic acid), preferably not more than 10 mol %, more preferably not more than 5 mol % and very preferably in the absence, of esters of the oligohydroxycarboxylic acid other than such esters of the oligohydroxycarboxylic acid with itself. Ruled out in particular is the presence of those esters of oligohydroxycarboxylic acid that carry free-radically polymerizable groups. This would have the disadvantage that the polymerization-sensitive free-radically polymerizable groups would be exposed during the esterification reaction to the reaction conditions of the esterification.

In a further preferred embodiment the reaction according to the invention is carried out in the absence of derivatives of the oligohydroxycarboxylic acid in which the hydroxyl groups have been provided with protective groups known to the skilled worker, more particularly in the absence of 5-alkyl-substituted 1,3-dioxane-5-carboxylic acids. These are reaction products of the oligohydroxycarboxylic acid with aldehydes or ketones, examples being acetonides.

These 5-alkyl-substituted 1,3-dioxane-5-carboxylic acids may be mono-, di- or unsubstituted in position 2, the alkyl group in position 5 preferably being a methyl or with particular preference an ethyl group.

Further protected derivatives of the oligohydroxycarboxylic acids in respect of which the reaction according to the invention is preferably performed in their absence are reaction products of the oligohydroxycarboxylic acid in which the hydroxyl groups have been connected via carbonate units or have been protected by silyl groups.

In another preferred embodiment, the reaction according to the invention is carried out in the absence of compounds comprising exclusively hydroxyl groups. Examples of compounds of this kind comprising exclusively hydroxyl groups are mono-, di- or polyalcohols, and also their esters with oligohydroxycarboxylic acids. A disadvantage of the presence of such alcohols is that these compounds can react with the carboxyl groups and would therefore lower the inventively desired carboxyl group content.

With regard to the preparation of the hyperbranched polyesters of the invention it is critical not to select too high a temperature during the reaction, since by this means the etherification reaction and/or lactone formation are/is promoted.

In general, the temperature during the reaction should be not more than 120° C., preferably not more than 110° C., more preferably not more than 100° C. and very preferably not more than 90° C.

It will be appreciated that a minimum temperature is needed in order for the reaction to proceed at a practical rate. In general, the temperature during the reaction should be at least 50° C., preferably at least 60° C. and more preferably at least 70° C.

The duration of the reaction, on the other hand, is generally of minor influence and is also dependent on the selected temperature. The higher the temperature, the shorter the reaction time that should be selected. The reaction time should not exceed 48 hours, preferably not more than 36 hours, with particular preference not more than 24 hours. Generally, however, at least 2 hours are needed, preferably at least 4 hours, more preferably at least 6 hours, very preferably at least 8 hours and more particularly at least 12 hours.

The critical factor is that, in the reaction, a sufficient conversion of the oligohydroxycarboxylic acid is ensured. The conversion, based on carboxyl groups reacted, ought to be at least 50%, preferably at least 66%, more preferably at least 75%, very preferably at least 80%, more particularly at least 85%, especially at least 90% and even at least 95%. It is also possible for even higher conversions to be sensible, such as at least 97%, 98% or even 99%.

The conversion can be monitored, for example, by monitoring the formation of the water which is released during the esterification reaction and which can be removed by distillation, for example, preferably by azeotropic distillation. Also conceivable, however, is to conduct the conversion until there is a significant increase in the viscosity of the reaction mixture.

To this end, the reaction is carried out in at least one, preferably precisely one, solvent. The solvent ought preferably to be inert under the reaction conditions; in other words it should not react with the reactant, intermediates or product. A solvent is considered inert if, under the reaction conditions, it is degraded by not more than 5% by weight, based on the initial quantity, in any way, such as by reaction with the reactant, intermediate or product, for example, or by thermal decomposition, and preferably is not degraded by more than 3% by weight, more preferably not more than 2%, very preferably not more than 1%, in particular not more than 0.5% and more particularly not more than 0.2% by weight.

Consequently, the use of alcohols or compounds containing carbonyl groups as solvents is less preferred. Alcohols, for example, are unsubstituted or substituted alkanols containing 1 to 12 carbon atoms, such as, for example, methanol, ethanol, isopropanol, n-propanol, n-butanol, isobutanol, sec-butanol, tert-butanol, ethylene glycol monomethyl ether, ethylene glycol monoethyl ether, n-hexanol, n-heptanol, n-octanol, n-decanol, n-dodecanol (lauryl alcohol), 2-ethylhexanol, cyclopentanol or cyclohexanol. Compounds containing carbonyl groups are, for example, aldehydes or, especially, ketones, examples being acetone, ethyl methyl ketone, diethyl ketone, isobutyl methyl ketone, cyclopentanone or cyclohexanone.

Conceivable, although less preferred, is the use of carboxylic esters as solvents, examples being n-butyl acetate, ethyl acetate, 1-methoxyprop-2-yl acetate and 2-methoxyethyl acetate.

The solvent employed ought to exhibit sufficient solubility for the oligohydroxycarboxylic acid. Preferred for this purpose, for example, are those solvents which are sufficiently polar and have a relative dielectric constant of 5 to 80, preferably 7 to 50 and more preferably 7.4 to 40.

Examples of such are halogenated hydrocarbons, preferably chlorinated hydrocarbons, examples being chlorinated aromatics or aliphatics. Preference is given to chlorobenzene, chlorotoluene, o-dichlorobenzene, methylene chloride, chloroform, 1,2-dichloroethane, 1,1-dichloroethane or 1,1,1-trichloroethane.

Also conceivable, however, are cyclic or acyclic ethers, examples being tetrahydrofuran, dioxane, tert-butyl methyl ether or tert-butyl ethyl ether.

There are in principle no upper limits on the boiling point of the solvent.

In order to allow easy distillative separation of the solvent employed, after the end of reaction, it is preferred to employ those solvents having a boiling point under atmospheric pressure of not more than 150° C.

It may additionally be of advantage to use solvents having an atmospheric-pressure boiling point which is at or above the reaction temperature. In that case the reaction can easily be carried out under atmospheric pressure or standard pressure and the solvent can easily be distilled off after the end of the reaction.

It is of course also conceivable to use solvents having a lower boiling point. In that case the reaction would have to be carried out under elevated pressure in order to achieve the required minimum temperature.

Additionally added to the reaction is at least one catalyst that accelerates the esterification reaction. Catalysts of this kind are known per se to the skilled worker.

The catalyst is preferably selected from the group consisting of acids, metal chelates, metal alkoxides, metal alkanoates and organometallic compounds.

Acids are preferably those having a pKa of less than 4.75, more preferably less than 4.0 and with particular preference less than 3.0.

As an acidic esterification catalyst it is preferred to use para-toluene sulfonic acid. Other acidic esterification catalysts that can be used are organic sulfonic acids, examples being methanesulfonic acid, benzenesulfonic acid, dodecylbenzenesulfonic acid, camphorsulfonic acid, cyclododecanesulfonic acid and/or sulfuric acid. Corresponding mixtures can be used as well. The amount of esterification catalyst, based on the reaction mixture comprised therein, is advantageously 0.1 to 10% by weight, preferably 0.1 to 6% by weight. Carboxylic acids in principle are likewise conceivable for use as catalysts, provided they have a sufficient acid strength. Since carboxylic acids, however, are themselves reactive and hence can additionally be included in the reaction, carboxylic acids are less preferred catalysts.

Metal compounds are alkanolates, alkanoates, chelates or organometallic compounds of the metals of groups IIIA to VIIIA or IB to VB in the Periodic Table of the Elements.

Preferred metals are boron, aluminum, tin, zinc, titanium, antimony, zirconium or bismuth.

Chelates are cyclic compounds in which metals and groups with lone electron pairs form a ring. A preferred chelating agent is acetylacetonate. An example of one such catalyst is zirconium acetylacetonate.

Alkanolates, for example, are C₁-C₁₀-alkanolates, preferably C₁-C₄-alkanolates, particular preference being given to methanolate, ethanolate, isopropanolate and n-butanolate, and very particular preference to methanolate and ethanolate and in particular to methanolate.

Alkanoates are, for example, C₁-C₂₀-alkanoates, preference being given to C₁-C₄-alkanoates, and particular preference to acetate.

Organometallic compounds are those having a direct metal-carbon bond.

Preferred metal compound catalysts are titanium tetrabutanolate, titanium tetraisopropanolate, zirconium tetrabutanolate, tin(II) n-octanoate, tin(II) 2-ethylhexanoate, tin(II) laurate, dibutyltin oxide, dibutyltin dichloride, dibutyltin diacetate, dibutyltin dilaurate, dibutyltin dimaleate, dioctyltin diacetate, antimony triethanolate or boronic acid derivatives, an example being pyridineboronic acid.

It is also possible to conceive of carrying out the reaction using one or more enzymes as catalysts. The use of lipases and esterases is preferred. Highly suitable lipases and esterases are Candida cylindracea, Candida lipolytica, Candida rugosa, Candida antarctica, Candida utilis, Chromobacterium viscosum, Geotrichum viscosum, Geotrichum candidum, Mucor javanicus, Mucor miehei, pig pancreas, Pseudomonas spp., Pseudomonas fluorescens, Pseudomonas cepacia, Rhizopus arrhizus, Rhizopus delemar, Rhizopus niveus, Rhizopus oryzae, Aspergillus niger, Penicillium roquefortii, Penicillium camembertii or esterase from Bacillus spp. and Bacillus thermoglucosidasius. Particular preference is given to Candida antarctica lipase B. The enzymes listed are available commercially, as for example from Novozymes Biotech Inc., Denmark.

One preferred embodiment may be that of modifying the hyperbranched polyesters of the invention with monofunctional acids, examples being carboxylic acids or sulfonic acids, preferably carboxylic acids. This modification may take place after the conclusion of the reaction of the oligohydroxycarboxylic acid or, preferably, during the reaction of the oligohydroxycarboxylic acid.

Additionally it is possible to use mixtures of two or more of the aforementioned acids. The acids may be used either as they are or in the form of derivatives. Such derivatives are more particularly
the anhydrides of said acids, in either monomeric or polymeric form;
the esters of said acids, e.g.,
   monoalkyl or dialkyl esters, preferably C₁ to C₄ alkyl esters, more preferably monomethyl or dimethyl esters, or the corresponding monoethyl or diethyl esters, but also the monoalkyl and dialkyl esters derived from higher alcohols such as, for example, n-propanol, isopropanol, n-butanol, isobutanol, tert-butanol, n-pentanol, n-hexanol,
   monovinyl and divinyl esters, and
   mixed esters, preferably methyl ethyl esters.

By way of example it is possible for the hyperbranched polymers to be reacted with alkyl- or alkenylcarboxylic acids, such as, for example, octanoic acid, nonanoic acid, decanoic acid, dodecanoic acid, hexadecanoic acid, stearic acid, oleic acid, linoleic acid, linolenic acid or the Li, Na, K, Cs, Ca or ammonium salts thereof, with alkylsulfonic acids, for example, octanesulfonic acid, dodecanesulfonic acid, stearylsulfonic acid or oleylsulfonic acid, or the Li, Na, K, Cs, Ca or ammonium salts thereof, with camphorsulfonic acid, cyclododecylsulfonic acid, p-toluenesulfonic acid, benzenesulfonic acid, 4-hexylbenzenesulfonate, 4-octylbenzenesulfonate, 4-decylbenzenesulfonate or 4-dodecylbenzenesulfonate or the Li, Na, K, Cs, Ca or ammonium salts thereof, with alkyl sulfates, for example, with n-alkyl sulfates or secondary alkyl sulfates.

The alkyl, alkenyl, cycloalkyl, or aryl radicals here may have up to 20 carbon atoms, preferably 6 to 20, more preferably 7 to 20.

Also conceivable, however, is the reaction with acids which comprise oxyalkylene groups. Preferably these are acids of the formula

R²—[—CH₂—CH₂—O]_{y}—R³—COOH

in which
R² is an alkyl group having 1 to 8 carbon atoms, preferably 1 to 4 carbon atoms,
y is 0 or a positive integer from 1 to 20, and
R³ is an alkylene group having 1 to 8, preferably 1 to 4, more preferably 1 to 2, and very preferably one carbon atom.

By an alkyl group having 1 to 8 carbons is meant methyl, ethyl, isopropyl, n-propyl, n-butyl, isobutyl, sec-butyl, tert-butyl, n-hexyl, n-heptyl, n-octyl, and 2-ethylhexyl.

By an alkylene group having 1 to 8 carbon atoms is meant methylene, 1,1-ethylene, 1,2-ethylene, 1,2-propylene, 1,3-propylene, 1,2-butylene, 1,3-butylene, 1,4-butylene, and 1,6-hexylene.

Preferably R² is methyl, ethyl or n-butyl, preferably methyl or n-butyl, and more preferably methyl.

Preferably y is a number from 1 to 10, more preferably from 1 to 5, and very preferably 1, 2 or 3.

Preferably R³ is methylene or 1,2-ethylene, more preferably methylene.

Of the stated monofunctional acids it is possible to react 0 to 40 mol %, preferably 0 to 30 mol %, more preferably 0 to 20 mol %, very preferably 0 to 10 mol %, and in particular 0 mol %, based on the oligohydroxycarboxylic acid used.

A further preferred embodiment may be that of modifying the hyperbranched polyesters of the invention with monohydroxycarboxylic acids or derivatives thereof. This modification may take place after the conclusion of the reaction of the oligohydroxycarboxylic acid or, preferably, during the reaction of the oligohydroxycarboxylic acid.

Derivatives in this case are as defined above and may additionally and preferably be lactones.

Examples of lactones in this context include gamma-butyrolactone, gamma-valerolactone, delta-valerolactone, gamma-caprolactone, and epsilon-caprolactone.

Examples of monohydroxycarboxylic acids are those of the formula

HO—R⁴—COOH

in which
R⁴ is an alkylene group having 1 to 8, preferably 2 to 6, more preferably 3 to 5, and very preferably 3 or 4 carbon atoms.

Preferably R⁴ is an α,ω-alkylene unit; conceivable, albeit less preferable, are α,(ω-1)- or α,(ω-2)-alkylene units.

Of the stated monohydroxycarboxylic acids it is possible to react 0 to 40 mol %, preferably 0 to 30 mol %, more preferably 0 to 20 mol %, very preferably 0 to 10 mol %, and in particular 0 mol %, based on the oligohydroxycarboxylic acid used.

The hyperbranched polyesters prepared in this way in accordance with the invention, and monofunctional with respect to the carboxylic acid group, are preferably reacted further to form hyperbranched polyesters of the kind in which the carboxyl function is derivatized with precisely one free-radically polymerizable group.

Examples of free-radically polymerizable groups are allyl groups, vinyl ether groups and preferably acrylate and methacrylate groups, the latter being referred to collectively for short in this specification as (meth)acrylate groups, and also double bonds conjugated in aromatic systems.

Allyl groups (H₂C═CH—CH₂—) can be present as they are or, preferably, in the form of allyl ether groups (H₂C═CH—CH₂—O—), although allyl ester groups (H₂C═CH—CH₂—O—CO—) are also conceivable.

Vinyl ether groups (H₂C═CH—O—) can also be present, alternatively, in the form of vinyl ester groups (H₂C═CH—O—CO—).

Examples of double bonds conjugated in aromatic systems are those of the kind present in styrene, α-methylstyrene, cinnamic acid or cinnamic esters.

The derivatization is accomplished by reacting the carboxyl group of the hyperbranched polyesters with at least one compound which carries at least one, preferably precisely one, carboxyl-reactive group and precisely one free-radically polymerizable group, preferably (meth)acrylate group.

Such compounds are preferably those of the formula

[RG]ₓ-R¹-Acr,

in which
x is a positive integer which is at least 1, preferably 1 to 3, more preferably 1 to 2 and very preferably precisely 1,
RG is a carboxyl-reactive group,
R¹ is an (x+1)-valent organic radical containing 1 to 20 carbon atoms, and
Acr is free-radically polymerizable group.

R¹ may be preferably C₆-C₁₂-arylene, C₃-C₁₂-cycloalkylene, C₁-C₂₀-alkylene or C₂-C₂₀-alkylene interrupted by one or more oxygen and/or sulfur atoms and/or by one or more substituted or unsubstituted imino groups and/or by one or more —(CO)—, —O(CO)O—, —(NH)(CO)O—, —O(CO)(NH)—, —O(CO)— or —(CO)O— groups.

C₃-C₁₂-cycloalkylene is for example cyclopropylene, cyclopentylene, cyclohexylene, cyclooctylene or cyclododecylene;

C₆-C₁₂-arylene is for example 1,2-, 1,3- or 1,4-phenylene, 2,3-, 2,4-, 2,5- or 2,6-tolylene, 1,8-, 1,5- or 2,6-naphthylene; and

C₁-C₂₀-alkylene is linear or branched alkylene, e.g. methylene, 1,2-ethylene, 1,2- or 1,3-propylene, 1,2-, 1,3- or 1,4-butylene, 1,1-dimethyl-1,2-ethylene or 1,2-dimethyl-1,2-ethylene, 1,6-hexylene, 1,8-octylene or 1,10-decylene.

There is no limit on the number of oxygen and/or sulfur atoms and/or imino groups in the C₂-C₂₀-alkylene. In general the number is not more than 5 in the radical, preferably not more than 4 and very preferably not more than 3.

Furthermore, there is generally at least one carbon atom located between two heteroatoms, and preferably there are at least two.

Possible examples of substituted and unsubstituted imino groups include imino, methylimino, isopropylimino, n-butylimino and tert-butylimino.

Examples of this kind of interrupted C₂-C₂₀-alkylene are 1-oxa-1,3-propylene, 1,4-dioxa-1,6-hexylene, 1,4,7-trioxa-1,9-nonylene, 1-oxa-1,4-butylene, 1,5-dioxa-1,8-octylene, 1-oxa-1,5-pentylene, 1-oxa-1,7-heptylene, 1,6-dioxa-1,10-decylene, 1-oxa-3-methyl-1,3-propylene, 1-oxa-3-methyl-1,4-butylene, 1-oxa-3,3-dimethyl-1,4-butylene, 1-oxa-3,3-dimethyl-1,5-pentylene, 1,4-dioxa-3,6-dimethyl-1,6-hexylene, 1-oxa-2-methyl-1,3-propylene, 1,4-dioxa-2,5-dimethyl-1,6-hexylene, 1-oxa-1,5-pent-3-enylene, 1-oxa-1,5-pent-3-ynylene, 1,1-, 1,2-, 1,3- or 1,4-cyclohexylene, 1,2- or 1,3-cyclopentylene, 1,2-, 1,3- or 1,4-phenylene, 4,4′-biphenylene, 1,4-diaza-1,4-butylene, 1-aza-1,3-propylene, 1,4,7-triaza-1,7-heptylene, 1,4-diaza-1,6-hexylene, 1,4-diaza-7-oxa-1,7-heptylene, 4,7-diaza-1-oxa-1,7-heptylene, 4-aza-1-oxa-1,6-hexylene, 1-aza-4-oxa-1,4-butylene, 1-aza-1,3-propylene, 4-aza-1-oxa-1,4-butylene, 4-aza-1,7-dioxa-1,7-heptylene, 4-aza-1-oxa-4-methyl-1,6-hexylene, 4-aza-1,7-dioxa-4-methyl-1,7-heptylene, 4-aza-1,7-dioxa-4-(2′-hydroxyethyl)-1,7-heptylene, 4-aza-1-oxa-(2′-hydroxyethyl)-1,6-hexylene or 1,4-piperazinylene.

R¹ here is preferably methylene, 1,2-ethylene, 1,2-propylene, 1,3-propylene, 1,4-butylene, 1,6-hexylene, 1,8-octylene or 1,10-decylene.

Examples of free-radically polymerizable groups Acr are allyl, methallyl, vinyl ether, acryloyl, methacryloyl and 2-phenylethen-1-yl (styrene) groups, preferably (meth)acrylate groups and more preferably acrylate groups.

Examples of carboxyl-reactive groups RG are primary amino groups, secondary amino groups, aziridines, azetidinium compounds and preferably epoxy groups.

These compounds preferably are the formal reaction products of epichlorohydrin (1-chloro-2,3-epoxypropane) with a compound which comprises precisely one free-radically polymerizable group and at least one, preferably one to three, more preferably one to two, and very preferably precisely one acid function.

Compounds of this kind which comprise precisely one free-radically polymerizable group and at least one acid function are, for example, acrylic acid, methacrylic acid, ethacrylic acid, α-chloroacrylic acid, crotonic acid, maleic acid, fumaric acid, itaconic acid, citraconic acid, mesaconic acid, glutaconic acid, aconitic acid, vinylsulfonic acid, vinylphosphonic acid, allylsulfonic acid, sulfoethyl acrylate, sulfoethyl methacrylate, 2- or 3-sulfopropyl acrylate, 2- or 3-sulfopropyl methacrylate, 2-hydroxy-3-acryloyloxy-propylsulfonic acid, 2-hydroxy-3-methacryloyloxypropylsulfonic acid, allylphosphonic acid, styrenesulfonic acid, cinnamic acid, 2-acrylamido-2-methylpropanesulfonic acid or 2-acrylamido-2-methylpropanephosphonic acid, or their amides, hydroxyalkyl esters and amino- or ammonio-containing esters and amides and, in the case of polybasic acids, the partly esterified compounds which still contain at least one carboxyl group.

Preference is given to acrylic acid, methacrylic acid, ethacrylic acid, crotonic acid, maleic acid, fumaric acid, itaconic acid, citraconic acid, mesaconic acid, glutaconic acid, aconitic acid, vinylsulfonic acid and cinnamic acid, particular preference to acrylic acid, methacrylic acid, crotonic acid, maleic acid and fumaric acid, very particular preference to acrylic acid and methacrylic acid, and more particularly to acrylic acid.

Examples of compounds which contain at least one, and precisely one free-radically polymerizable group are allyl glycidyl ether, vinyl glycidyl ether, glycidyl acrylate and glycidyl methacrylate, preferably glycidyl acrylate and glycidyl methacrylate.

Reaction of the carboxyl group of the hyperbranched polyester with the compound which contains at least one carboxyl-reactive group and precisely one free-radically polymerizable group takes place, for example, under the following reaction conditions:

The stoichiometry of the reaction is for example 0.75 to 1.25, preferably 0.8 to 1.2, more preferably 0.9 to 1.1 and very preferably 0.95 to 1.05 mol of carboxyl-reactive groups per mole of carboxyl groups in the hyperbranched polyester (which can be determined, for example, via its acid number).

For the reaction it is possible for both components or preferably the hyperbranched polyester to be dissolved optionally in a solvent. Suitable solvents are the same solvents as described above for the esterification reaction of the oligohydroxycarboxylic acids. Here, however, alcohols and solvents containing carbonyl groups are equally preferred.

The solvent employed ought to have sufficient solubility for the reaction components. Preference is given for this purpose, for example, to solvents which are sufficiently polar and have a relative dielectric constant of 5 to 80, preferably 7 to 50 and more preferably 7.4 to 40.

Preference is given to polar aprotic solvents, and particular preference to dimethyl-formamide, dimethylacetamide, acetonitrile and dimethyl sulfoxide.

The hyperbranched polyester, in solution in a solvent, is usually introduced as the initial charge, and the compound which contains at least one carboxyl-reactive group and precisely one free-radically polymerizable group is added in portions.

The reaction temperature can be from 50 to 120° C., preferably 60 to 100° C., more preferably 70 to 90° C.

The duration of the reaction is dependent on the temperature selected. Generally speaking, 24 hours are sufficient, preferably less than 18, and with particular preference less than 14 hours. In general, however, at least one hour is necessary, preferably at least 2 hours, more preferably at least 4 hours, very preferably at least 6 hours, and in particular at least 10 hours.

To accelerate the reaction it can be an advantage to add at least one catalyst. Examples of such are amines, tertiary amines being preferred. Tributylamine is particularly preferred.

The present invention also provides for the use of the high-functionality, highly branched, hyperbranched polyesters of the invention which carry a polymerizable group as a monomer or comonomer in free-radical polymerization. The polymers or copolymers obtained from such a (co)polymerization are suitable, for example, for use in coating systems, particularly in what are called dual-cure coating systems, in which the curing takes place via different curing mechanisms—hence in this case, for example, by free-radical curing of the free-radically polymerizable group and by reaction of the hydroxyl groups with polyisocyanates.

### EXAMPLES

### Example 1

### Reaction of dimethylolbutyric acid in 1,2-dichloroethane, Catalyzed with para-toluenesulfonic acid

In a 4 l three-necked flask equipped with water separator, internal thermometer and stirrer, 250 g of dimethylolbutyric acid (DMBA) were admixed with 1.4 l of 1,2-dichloroethane and 5.0 g of para-toluenesulfonic acid were added as a catalyst. The reaction mixture was then heated under reflux (about 83° C.) and the water formed during the esterification was removed via the water separator. After a reaction time of 19 h a total of 24.6 g of water were removed, corresponding to a conversion of approximately 83%. In the course of the reaction, the polymeric product is deposited as a white solid on the wall of the flask. Subsequently the solvent, 1,2-dichloroethane, is removed by filtration and/or distillation.

The number-average molar weight Mₙ of the polyester prepared was determined by means of GPC (eluent: hexafluoroisopropanol [HFIP], calibration: PMMA (polymethyl methacrylate)) to be 1100 g/mol, the weight-average molar weight M_{w} to be 2200 g/mol. The acid number was determined in accordance with DIN 53240, Part 2, to be 43 mg KOH/g polymer.

### Example 1a

In an analogous comparative example to Example 1, a total of 20.5 g of water were removed, corresponding to a lower conversion of approximately 67%. The polymeric product here again was deposited as a white solid on the wall of the flask in the course of the reaction. Subsequently the solvent, 1,2-dichloroethane, was removed by filtration and/or distillation.

The number-average molar weight Mₙ of the polyester prepared was determined by means of GPC (eluent: hexafluoroisopropanol [HFIP], calibration: PMMA) to be 1100 g/mol, the weight-average molar weight M_{w} to be 1700 g/mol.

### Example 2

### (Comparative to Example 1): Reaction of dimethylolbutyric acid in dichloromethane, Catalyzed with para-toluenesulfonic acid

In a 4 l three-necked flask equipped with water separator, internal thermometer and stirrer, 250 g of dimethylolbutyric acid (DMBA) were admixed with 1.4 l of dichloromethane and 5.0 g of para-toluenesulfonic acid were added as a catalyst. The reaction mixture was then heated under reflux (about 40° C.) and the water formed during the esterification was removed via the water separator. Here, it is found that the reaction rate at 40° C. is too low to allow any significant removal of water from the reaction mixture.

### Example 3

### (Comparative to Example 1): Reaction of dimethylolbutyric acid in 1,1,2,2-tetrachloromethane, Catalyzed with para-toluenesulfonic acid

In a 4 l three-necked flask equipped with water separator, internal thermometer and stirrer, 100 g of dimethylolbutyric acid (DMBA) were admixed with 1.4 l of 1,1,2,2-tetrachloroethane and 2.0 g of para-toluenesulfonic acid were added as a catalyst. The reaction mixture was then heated under reflux (about 147° C.) and the water formed during the esterification was removed via the water separator. After a reaction time of 12 h a total of 26.3 g of water were removed, corresponding to a conversion of approximately 88%. Subsequently the solvent, 1,1,2,2-tetrachloroethane, is removed by distillation. The product comprises significant fractions of components having more than one COOH group per molecule.

### Example 4

### (Comparative to Example 1): Reaction of dimethylolbutyric acid in acetone, Catalyzed with titanium(IV) butoxide

In a 4 l three-necked flask equipped with top-mounted distillation attachment, consisting of a descending condenser and a reservoir flask, and equipped with internal thermometer and stirrer, 151 g of dimethylolbutyric acid (DMBA) were admixed with 0.2 g of titanium(IV) butoxide as catalyst, dissolved in anhydrous acetone, and slowly heated. Acetone was distilled off continuously from the reaction mixture, and serves as an azeotrope former for the water formed as a byproduct of the condensation. In order to compensate this loss of solvent, new anhydrous acetone was added continually in parallel to the mixture, in the appropriate quantity. The progress of the reaction was monitored by determining the water content of the individual acetone distillates by means of Karl-Fischer titration. After a reaction time of about 72 hours a total of about 15.6 g of water was removed, corresponding to a conversion of approximately 85%. The product comprises predominantly components with acetonide-protected diol units, which would first have to be eliminated again in a further reaction step, which would entail additional, unnecessary cost and inconvenience.

### Example 5

### (Comparative to Example 1): Reaction of dimethylolbutyric acid without solvent, Catalyzed with para-toluenesulfonic acid

In a 4 l three-necked flask equipped with top-mounted distillation attachment, consisting of a short Vigreux column, a descending condenser and a reservoir flask, and also equipped with internal thermometer and stirrer, 100 g of dimethylolbutyric acid (DMBA) were admixed with 2.0 g of para-toluenesulfonic acid as catalyst, and slowly heated. In the course of this heating the reaction mixture was heated up to 220° C. and the water formed in the esterification was removed via the distillation apparatus. After a reaction time of about 3 h, a total of 10.1 g of water was removed, corresponding to a conversion of approximately 82%. The product comprises significant fractions of components having more than one COOH group per molecule.

### Example 6

### (Comparative to Example 1): Reaction of dimethylolbutyric acid without solvent, Catalyzed with titanium(IV) butoxide

In a 4 l three-necked flask equipped with top-mounted distillation attachment, consisting of a short Vigreux column, a descending condenser and a reservoir flask, and also equipped with internal thermometer and stirrer, 100 g of dimethylolbutyric acid (DMBA) were admixed with 0.2 g of titanium(IV) butoxide as catalyst, and slowly heated. In the course of this heating the reaction mixture was heated up to 220° C. and the water formed in the esterification was removed via the distillation apparatus. After a reaction time of 1.5 h, a total of 10.2 g of water was removed, corresponding to a conversion of approximately 83%. The product comprises significant fractions of components having more than one COOH group per molecule.

### Example 7

### Reaction of dimethylolbutyric acid and dimethylolpropionic acid in 1,2-dichloroethane, Catalyzed with para-toluenesulfonic acid

In a 4 l three-necked flask equipped with water separator, internal thermometer and stirrer, 132 g of dimethylolbutyric acid (DMBA) and 118 g of dimethylolpropionic acid (DMPA) were admixed with 2.0 l of 1,2-dichloroethane and 5.0 g of para-toluene-sulfonic acid were added as a catalyst. The reaction mixture was then heated under reflux (about 84° C.) and the water formed during the esterification was removed via the water separator. After a reaction time of 20 h a total of 27.5 g of water were removed, corresponding to a conversion of approximately 90%. In the course of the reaction, the polymeric product was deposited as a white solid on the wall of the flask. Subsequently the solvent, 1,2-dichloroethane, was removed by filtration and/or distillation. The number-average molar weight Mₙ of the polyester prepared was determined by means of GPC (eluent: hexafluoroisopropanol [HFIP], calibration: PMMA) to be 2200 g/mol, the weight-average molar weight M_{w} to be 4200 g/mol. The acid number was determined in accordance with DIN 53240, Part 2, to be 24 mg KOH/g polymer.

### Example 8

### Reaction of dimethylolpropionic acid in 1,2-dichloroethane, Catalyzed with para-toluenesulfonic acid

In a 4 l three-necked flask equipped with water separator, internal thermometer and stirrer, 250 g of dimethylolpropionic acid (DMPA) were admixed with 2.0 l of 1,2-dichloroethane and 5.0 g of para-toluenesulfonic acid were added as a catalyst. The reaction mixture was then heated under reflux (about 84° C.) and the water formed during the esterification was removed via the water separator. After a reaction time of 36 h a total of 32.0 g of water were removed, corresponding to a conversion of approximately 96%. In the course of the reaction, the polymeric product was deposited as a white solid on the wall of the flask. Subsequently the solvent, 1,2-dichloroethane, was removed by filtration and/or distillation.

The number-average molar weight Mₙ of the polyester prepared was determined by means of GPC (eluent: hexafluoroisopropanol [HFIP], calibration: PMMA) to be 5400 g/mol, the weight-average molar weight M_{w} to be 15 400 g/mol. The acid number was determined in accordance with DIN 53240, Part 2, to be 10 mg KOH/g polymer.

### Example 9

### Reaction of dimethylolbutyric acid and isononanoic acid in 1,2-dichloroethane, Catalyzed with para-toluenesulfonic acid

In a 4 l three-necked flask equipped with water separator, internal thermometer and stirrer, 236 g of dimethylolbutyric acid (DMBA) and 24.5 g of isononanoic acid were admixed with 2.0 l of 1,2-dichloroethane and 5.0 g of para-toluenesulfonic acid were added as a catalyst. The reaction mixture was then heated under reflux (about 85° C.) and the water formed during the esterification was removed via the water separator. After a reaction time of 48 h a total of 18.4 g of water were removed, corresponding to a conversion of approximately 60%. Subsequently the solvent, 1,2-dichloroethane, was removed by filtration and/or distillation.

### Example 10

### Reaction of dimethylolbutyric acid and isononanoic acid in 1,2-dichloroethane, Catalyzed with para-toluenesulfonic acid

In a 4 l three-necked flask equipped with water separator, internal thermometer and stirrer, 236 g of dimethylolbutyric acid (DMBA) and 49.0 g of isononanoic acid were admixed with 2.0 l of 1,2-dichloroethane and 5.0 g of para-toluenesulfonic acid were added as a catalyst. The reaction mixture was then heated under reflux (about 84° C.) and the water formed during the esterification was removed via the water separator. After a reaction time of 24 h a total of 21.0 g of water were removed, corresponding to a conversion of approximately 63%. Subsequently the solvent, 1,2-dichloroethane, was removed by filtration and/or distillation.

### Example 11

### Reaction of dimethylolbutyric acid and γ-butyrolactone in 1,2-dichloroethane, Catalyzed with para-toluenesulfonic acid

In a 4 l three-necked flask equipped with water separator, internal thermometer and stirrer, 236 g of dimethylolbutyric acid (DMBA) and 13.5 g of γ-butyrolactone were admixed with 2.0 l of 1,2-dichloroethane and 5.0 g of para-toluenesulfonic acid were added as a catalyst. The reaction mixture was then heated under reflux (about 84° C.) and the water formed during the esterification was removed via the water separator. After a reaction time of 48 h a total of 17.5 g of water were removed, corresponding to a conversion of approximately 63%. Subsequently the solvent, 1,2-dichloroethane, was removed by filtration and/or distillation.

### Example 12

### Reaction of dimethylolbutyric acid and 2-[2-(2-methoxy-ethoxy)ethoxy]acetic acid in 1,2-dichloroethane, Catalyzed with para-toluenesulfonic acid

In a 4 l three-necked flask equipped with water separator, internal thermometer and stirrer, 756 g of dimethylolbutyric acid (DMBA) and 198 g of 2-[2-(2-methoxy-ethoxy)ethoxy]acetic acid were admixed with 2.0 l of 1,2-dichloroethane and 5.0 g of para-toluenesulfonic acid were added as a catalyst. The reaction mixture was then heated under reflux (about 84° C.) and the water formed during the esterification was removed via the water separator. After a reaction time of 25 h a total of 93.5 g of water were removed, corresponding to a conversion of approximately 87%. Subsequently the solvent, 1,2-dichloroethane, was removed by filtration and/or distillation.

### Example 13

### Reaction of the polyester from Example 1 with glycidyl methacrylate

320 g of a solution of the polyester from Example 1 in dimethylformamide (DMF) (16% by weight) were dried with 50 g of molecular sieve and then introduced into a 1 l three-necked flask equipped with stirrer, internal thermometer and reflux condenser. Thereafter, corresponding to the number of acid groups in the polyester, 14.8 g of glycidyl methacrylate (1 eq. relative to the number of COOH groups), tributylamine catalyst (0.97 g), and the stabilizers hydroquinone monomethyl ether (MEHQ, 0.03 g) and 2,6-di-tert-butyl-4-methylphenol (DBPC, 0.03 g) were added. The mixture was stirred at 80° C. for 12 h. Thereafter the solvent was removed on a rotary evaporator. The number-average molar weight Mₙ of the polyester prepared, comprising a polymerizable group, was determined by means of GPC (eluent: hexafluoroisopropanol [HFIP], calibration: PMMA) to be 1100 g/mol, the weight-average molar weight M_{w} to be 5000 g/mol. The acid number was determined in accordance with DIN 53240, Part 2, to be 0.1 mg KOH/g polymer.

### Example 14

### Reaction of the polyester from Example 7 with glycidyl methacrylate

325 g of a solution of the polyester from Example 7 in dimethylformamide (DMF) (10% by weight) were dried with 80 g of molecular sieve and then introduced into a 1 l three-necked flask equipped with stirrer, internal thermometer and reflux condenser. Thereafter, corresponding to the number of acid groups in the polyester, 2.2 g of glycidyl methacrylate (1.1 eq. relative to the number of COOH groups), tributylamine catalyst (0.52 g), and the stabilizers hydroquinone monomethyl ether (MEHQ, 0.02 g) and 2,6-di-tert-butyl-4-methylphenol (DBPC, 0.02 g) were added. The mixture was stirred at 80° C. for 16 h. Thereafter the solvent was removed on a rotary evaporator. The number-average molar weight Mₙ of the polyester prepared was determined by means of GPC (eluent: hexafluoroisopropanol [HFIP], calibration: PMMA) to be 2300 g/mol, the weight-average molar weight M_{w} to be 4600 g/mol. The acid number was determined in accordance with DIN 53240, Part 2, to be 0.1 mg KOH/g polymer.

### Example 15

### Reaction of the polyester from Example 8 with glycidyl methacrylate

113 g of a solution of the polyester from Example 8 in dimethylformamide (DMF) (10% by weight) were dried with 30 g of molecular sieve and then introduced into a 1 l three-necked flask equipped with stirrer, internal thermometer and reflux condenser. Thereafter, corresponding to the number of acid groups in the polyester, 0.32 g of glycidyl methacrylate (1.1 eq. relative to the number of COOH groups), tributylamine catalyst (0.17 g), and the stabilizers hydroquinone monomethyl ether (MEHQ, 0.01 g) and 2,6-di-tert-butyl-4-methylphenol (DBPC, 0.01 g) were added. The mixture was stirred at 80° C. for 16 h. Thereafter the solvent was removed on a rotary evaporator. The number-average molar weight Mₙ of the polyester prepared was determined by means of GPC (eluent: hexafluoroisopropanol [HFIP], calibration: PMMA) to be 5000 g/mol, the weight-average molar weight M_{w} to be 17 700 g/mol. The acid number was determined in accordance with DIN 53240, Part 2, to be 0.0 mg KOH/g polymer.

1. A hyperbranched polyester having a weight-average molecular weight MW between 500 and 300 000 g/mol, obtained by reacting at least one oligohydroxycarboxylic acid in the presence of at least one catalyst and in the presence of at least one solvent inert under the reaction conditions, wherein the temperature during the reaction is set at not more than 120° C., the average carboxyl functionality per molecule is more than 0.8 and less than 2, and the average ether group functionality —CH₂—O—CH₂ is less than 0.20.

2. The hyperbranched polyester according to claim 1, wherein additionally the average lactone functionality is less than 0.10.

3. The hyperbranched polyester according to claim 1, wherein the oligohydroxycarboxylic acid is a dihydroxyalkanecarboxylic acid.

4. The hyperbranched polyester according to claim 1, wherein the oligohydroxycarboxylic acid is a 2,2-bis(hydroxymethyl)alkanecarboxylic acid.

5. The hyperbranched polyester according to claim 1, wherein the reaction is carried out in the absence of esters of the oligohydroxycarboxylic acid except esters of the oligohydroxycarboxylic acid with itself.

6. The hyperbranched polyester according to claim 1, wherein the reaction is carried out in the absence of 5-alkyl-substituted 1,3 dioxane-5-carboxylic acids.

7. The hyperbranched polyester according to claim 1, wherein the reaction is carried out in the absence of compounds comprising exclusively hydroxyl groups.

8. The hyperbranched polyester according to claim 1, wherein the oligohydroxycarboxylic acid is dimethylolpropionic acid.

9. The hyperbranched polyester according to claim 1, wherein the oligohydroxycarboxylic acid is dimethylolbutyric acid.

10. The hyperbranched polyester according to claim 1, wherein the oligohydroxycarboxylic acid is selected from the group consisting of 3,5 dihydroxybenzoic acid and 4,4-bis(4-hydroxyphenyl)valeric acid.

11. The hyperbranched polyester according to claim 1, wherein the solvent has a boiling point under atmospheric pressure of not more than 150° C.

12. The hyperbranched polyester according to claim 1, wherein the solvent has a relative dielectric constant of 5 to 80.

13. The hyperbranched polyester according to claim 1, wherein the catalyst is an acid having a pKa of less than 4.75.

14. The hyperbranched polyester according to claim 1, wherein the catalyst is an organometallic compound.

15. The hyperbranched polyester according to claim 1, wherein the catalyst is an enzyme.

16. The hyperbranched polyester according to claim 1, wherein the reaction takes places in the presence of monofunctional acids or derivatives thereof.

17. The hyperbranched polyester according to claim 1, wherein, after the conclusion of the reaction of the oligohydroxycarboxylic acid, a reaction with monofunctional acids takes place.

18. The hyperbranched polyester according to claim 16, wherein the monofunctional acid is a carboxylic acid or sulfonic acid.

19. The hyperbranched polyester according to claim 16, wherein the monofunctional acid is an alkyl- or alkenylcarboxylic acids whose alkyl or alkenyl radical comprises up to 20 carbon atoms.

20. The hyperbranched polyester according to claim 16, wherein the monofunctional acid has the formula
R²—[—CH₂—CH₂—O]_{y}—R³—COOH
wherein
R² is an alkyl group having 1 to 8 carbon atoms,
y is 0 or a positive integer from 1 to 20, and
R³ is an alkylene group having 1 to 8 carbon atoms.

21. The hyperbranched polyester according to claim 1, wherein the reaction takes place in the presence of monohydroxycarboxylic acids or derivatives thereof.

22. The hyperbranched polyester according to claim 21, wherein the monohydroxy-carboxylic acid is of the formula
HO—R⁴—COOH
wherein
R⁴ is an alkylene group having 1 to 8.

23. A free-radically polymerizable hyperbranched polyester wherein a hyperbranched polyester according to claim 1 is reacted with at least one compound which carries at least one carboxyl-reactive group and precisely one free-radically polymerizable group.

24. The free-radically polymerizable, hyperbranched polyester according to claim 23, wherein the free-radically polymerizable group is selected from the group consisting of allyl, methallyl, vinyl ether, acryloyl, methacryloyl and 2 phenylethen-1-yl (styrene) groups.

25. The free-radically polymerizable, hyperbranched polyester according to claim 23, wherein the compound which carries at least one carboxyl-reactive group and precisely one free-radically polymerizable group is the formal reaction product of epichlorohydrin (1-chloro-2,3-epoxypropane) with a compound which comprises precisely one free-radically polymerizable group and at least one acid function.

26. The free-radically polymerizable, hyperbranched polyester according to claim 25, wherein the compound which comprises precisely one free-radically polymerizable group and at least one acid function is acrylic acid, methacrylic acid, ethacrylic acid, α-chloroacrylic acid, crotonic acid, maleic acid, fumaric acid, itaconic acid, citraconic acid, mesaconic acid, glutaconic acid, aconitic acid, vinylsulfonic acid, vinylphosphonic acid, allylsulfonic acid, sulfoethyl acrylate, sulfoethyl methacrylate, 2- or 3-sulfopropyl acrylate, 2- or 3-sulfopropyl methacrylate, 2-hydroxy-3-acryloyloxypropylsulfonic acid, 2-hydroxy-3-methacryloyloxypropylsulfonic acid, allylphosphonic acid, styrenesulfonic acid, cinnamic acid, 2-acrylamido-2-methylpropanesulfonic acid or 2-acrylamido 2 methylpropanephosphonic acid, or one of their amides, hydroxyalkyl esters and amino- or ammonio-containing esters and amides and, in the case of polybasic acids, the partly esterified compounds which comprise at least one carboxyl group.

27. The free-radically polymerizable, hyperbranched polyester according to claim 23, wherein the compound which carries at least one carboxyl-reactive group and precisely one free-radically polymerizable group is allyl glycidyl ether, vinyl glycidyl ether, glycidyl acrylate or glycidyl methacrylate.

28. A process for preparing a free-radically polymerizable, hyperbranched polyester according to claim 23, comprising reacting at least one hyperbranched polyester with at least one compound which carries at least one carboxyl-reactive group and precisely one free-radically polymerizable group.

29. A monomer or comonomer infra-radical polymerization comprising a free-radically polymerizable, high-functionality, highly branched, hyperbranched polyester according to claim 23.

30. (canceled)
